(19) United States
(12) Patent Application Publication
(10) Pub. No.: US 2004/0029959 A1
(43) Pub. Date: Feb. 12, 2004

(54) Isosorbide mononitrate compositions and methods of their use

(76) Inventors: John Devane, Athlone (IE); Paul Stark, Athlone (IE); John Kelly, Dublin (IE); Jackie Butler, Athlone (IE)

Correspondence Address: CROWELL AND MORING , LLP, PO BOX 14300, WASHINGTON, DC, 20044-4300, US

(21) Appl. No.: 10/214,345

(22) Filed: Aug. 8, 2002

(51) Int. Cl. A61K 31/34; A61K 9/22

(52) U.S. Cl. 514/471

(57) ABSTRACT

The present invention relates to delayed onset, extended release formulations of isosorbide mononitrates (ISMNs), and methods of their use in treating, preventing, reducing, reversing, and/or managing nitrate tolerance and/or cardiovascular conditions. In particular, the present invention is directed to once-daily delayed onset, extended release formulations that (1) provide a subject with a therapeutically effective amount of ISMNs during the early morning hours prior to and after awakening, (2) continue to provide therapeutically effective amounts of ISMN throughout the waking hours of the day, and (3) provide a reduction, or washout, of ISMN plasma levels to treat, prevent, reduce, reverse, and/or manage nitrate tolerance.

Isosorbide mononitrates (ISMNS) are vasodilators that are able to reduce myocardial oxygen demands while maintaining or increasing coronary artery flow. Due to their biological activity, doctors often prescribe ISMNs to treat cardiovascular conditions, such as angina pectoris. Two common isosorbide mononitrates are isosorbide-5-mononitrate (IS-5-MN) and isosorbide-2-mononitrate (IS-2-MN).

Unlike isosorbide dinitrates (ISDN), ISMNs do not undergo substantial first pass liver metabolism. Thus, ISMNs provide a greater bioavailability relative to ISDNs. In addition, ISMNs are more completely absorbed from the gastrointestinal tract after oral administration and have a much longer half-life than ISDNs (Straehl et al., Clin. Pharmacol. Ther., 36:485-92, 1984).

Despite the advantages of ISMNs, there are significant limitations to their use. Regular administration of nitrates, including ISMNS, in which plasma nitrate concentrations are maintained during a 24 hour period, causes subjects to rapidly develop a tolerance to the presence of nitrate. Nitrate tolerance is characterized by a loss or significant reduction in the responsiveness of the target tissue to the nitrate being administered. While the direct cause of nitrate tolerance is still a matter of some speculation, it may result from alterations in the target tissues (e.g., the arterial and venous smooth muscle), making the tissues less sensitive or refractory to the effects of nitrates. The phenomenon of nitrate tolerance has been observed in humans with all commonly used nitrates, regardless of the method or route of administration. Nitrate tolerance significantly reduces the biological efficacy of nitrate therapy (see, e.g., Thadani, Cardiovasc. Drugs Ther., 10(6):735-42, 1997).

Conventional (e.g., 10-50 mg two to three times daily), extended release (e.g., 20-240 mg one time daily) ISMN formulations generally achieve an initial effect, but the magnitude and duration of that effect is reduced by tolerance that develops over the course of therapy. Tolerance develops not only with a single daily treatment, but also with repeated administrations. Therefore, to maintain a therapeutic effect, the plasma concentration should be increased throughout the day, or each successive dosage must be gradually increased. But this requires continual monitoring and alterations in the dosing regimen to safely manage the subject, which is clearly impracticable.

Studies suggest that short periods of nitrate withdrawal, typically less than 12 hours, may prevent the effects of tolerance and maintain the therapeutic efficacy of nitrates. Accordingly, attempts have been made to prevent nitrate tolerance by incorporating a “washout” phase into treatment regimens. During a washout phase, the plasma concentration of nitrate is allowed to drop below a therapeutically effective level for a specified length of time. Following the washout, a subject receives a dose of nitrate sufficient to restore therapeutic levels. This is followed by a subsequent washout, and the cycle of therapy is repeated.

With conventional oral nitrate formulations, administered two or three times per day, a washout can be achieved by simply omitting the final dose. In the case of a transdermal device, the device can be removed after 12 or more hours, ceasing the delivery of the nitrate therapy and allowing the plasma concentration of the nitrate to drop to sub-therapeutic levels.

Once daily formulations are desirable because patient compliance can be as high as 80%, while with twice-a-day and three times-a-day dosing, compliance levels fall to 60% and 40%, respectively (see, e.g., Shilo, et al., Ann. Pharmacother., 35(11):1339-42, 2001). Thus, dosage forms that reduce the frequency of administration can significantly improve the therapeutic outcome. A washout phase cannot be achieved, however, by simply omitting a dose with such once-a-day formulations. Instead, the single dose must be formulated to provide the desired pharmacokinetic profile, achieving a sufficient duration of therapeutic levels throughout the day, while also providing for a washout phase to treat, prevent, reduce, reverse, and/or manage tolerance.

In addition to providing therapeutic levels of nitrates throughout the day and treating, preventing, reducing, reversing, and/or managing the problems of tolerance, advantageous ISMN formulations desirably relieve the early morning pathologies reported by patients suffering from cardiovascular conditions such as angina. It is well-documented that there is an increased risk for these patients to experience sudden death, myocardial infarction, and acute cerebrovascular events in the morning hours. Additionally, these patients often experience discomfort just before, and for the first few hours after, awakening. To avoid or relieve these symptoms, nitrate formulations should provide a patient with a therapeutically effective amount of nitrate just prior to awakening, and during the early morning hours.

Busetti (U.S. Pat. Nos. 5,788,987; 5,891,474; 6,190,692) describes a delayed-release formulation that, when administered prior to sleep, produces a pharmaceutically effective concentration of an active compound at about the time of awakening. The formulation is prepared by coating a drug core with a swellable polymer; the length of the delay in release of the drug depends on the thickness of the polymeric coating. After the delay period, during which the polymeric coating is removed by dissolution or erosion, the active compound is exposed and rapidly released into the patient's system.

When applied to nitrate therapy, this type of rapid release provides an initial spike followed by a rapid decline in the blood plasma concentration levels of the nitrate. Thus, while nitrate may be present at therapeutic levels during the early morning hours (e.g., during the spike), this level is not maintained throughout the waking hours of the day. Consequently, this approach to therapy does not provide a patient with adequate protection during the day. Busetti does not describe a dosage form that achieves a delayed and extended release of an active compound, providing a therapeutic benefit beyond the early morning hours and throughout the day.

Bayer (U.S. Pat. No. 4,956,181) describes a treatment for morning pathologies associated with angina that involves the delivery of nitrates in a delayed-release transdermal patch. The delivery of the nitrate is initially retarded by a polymeric physical barrier, which becomes permeable to the drug after the delay period. Following the delay period, the drug is rapidly released into the patient at an ever-increasing rate. By applying the patch at bedtime, an initial effective delivery rate is reportedly achieved about 45 to 90 minutes before awakening. The rate reportedly increases from about 125% to 1000% of the initial delivery rate over the course of the next 8 to 21 hours. Bayer indicates that such a release profile is contrary to those achieved in other transdermal systems, which typically provide substantially uniform delivery rates. The therapy is ended when the drug is exhausted or the patch is removed. Bayer briefly mentions that its transdermal treatment may be provided as an oral delivery system. Bayer, however, does not teach any formulation suitable for oral administration, or an oral formulation that would exhibit the pharmacokinetics suitable for treating morning pathologies.

Thus, there exists a need in the art for new methods of once-daily administration of nitrates, and formulations for use in such methods, that can treat morning pathologies and continue to provide therapeutically effective amounts of nitrate throughout the day, while treating, preventing, reducing, reversing, and/or managing nitrate tolerance that is associated with conventional nitrate therapy.

### BRIEF DESCRIPTION OF THE FIGURES

FIG. 1 illustrates the results observed for a sustained release tablet formulation administered in the morning (at about 8 AM), or at night (at about 10 PM).

FIG. 2 shows the results observed for a sustained release tablet, prepared as described in Example 1, administered at night in either coated or uncoated form.

FIG. 3 illustrates the dissolution profiles for different IS-5-MN (60 mg) delayed onset, extended release oral dosage forms.

FIG. 4 compares the blood plasma concentration of IS-5-MN following administration of three formulations of IS-5-MN (60 mg) delayed onset, extended release oral dosage forms and IMDUR™ (Key Pharmaceuticals).

### DEFINITIONS

As used herein, the phrase “delayed release” formulation refers to a pharmaceutical preparation that substantially or completely withholds or impairs delivery of a compound for a specified period of time, i.e., the delay period. Following this delay period, the active ingredient of such formulations begins to be released. Without further impairment, the full amount of the drug is released rapidly. For example, a typical delayed-release tablet will inhibit release of its active compound until an exterior coating disintegrates or erodes. Then, once the coating is dissolved, the active compound is rapidly released into the patient.

As used herein, the term “ISMN” includes all isosorbide mononitrates, and any pharmaceutically acceptable salts thereof.

As used herein, the term “pharmaceutically acceptable excipient” includes compounds that are compatible with the other ingredients in a pharmaceutical formulation and not injurious to the subject when administered in acceptable amounts.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

As used herein, the phrase “therapeutically effective amount” includes the amount of nitrate (or pharmaceutically acceptable salt thereof), which alone or in combination with other nitrates and/or drugs, provides a benefit in treating, preventing, reducing, reversing, and/or managing one or more cardiovascular conditions that may benefit from the properties of nitrates as relaxants of smooth muscle, and/or as dilators of blood vessels. Such conditions include, but are not limited to, angina pectoris, congestive heart failure and myocardial infarction. In one embodiment, the cardiovascular condition is angina pectoris and/or congestive heart failure.

As used herein, the phrase “extended release” formulation or dosage form includes a pharmaceutical preparation that maintains a therapeutically effective level of an active compound in a subject for a specified period of time. In addition to maintaining therapeutic levels of the active compound, an extended release formulation may also be designed to delay the release of the active compound for a specified period of time. Such compounds are referred to herein as “delayed onset, extended release” formulations or dosage forms.

### DESCRIPTION OF THE INVENTION

The present invention relates to delayed onset, extended release formulations comprising one or more isosorbide mononitrates (ISMNs), and methods of their use in treating, preventing, reducing, reversing, and/or managing nitrate tolerance and/or cardiovascular conditions. In particular, the present invention is directed to once-daily delayed onset, extended release formulations, and methods of their use, that (1) provide a subject with a therapeutically effective amount of one or more ISMNs during the early morning hours prior to and after awakening, (2) continue to provide therapeutically effective amounts of one or more ISMNs throughout the waking hours of the day, and (3) provide a reduction, or washout, of ISMN plasma levels to treat, prevent, reduce, reverse, and/or manage nitrate tolerance.

The compositions and methods of the present invention are particularly useful in treating, preventing, reducing, reversing, and/or managing nitrate tolerance and cardiovascular conditions. Cardiovascular conditions that may be treated with the present methods and compositions include conditions that may benefit from the properties of nitrates as relaxants of smooth muscle, and as dilators of blood vessels. Such conditions include, but are not limited to, angina pectoris, congestive heart failure and myocardial infarction. In one embodiment, the cardiovascular condition is angina pectoris and/or congestive heart failure.

The present ISMN delayed onset, extended release formulations, and methods of their use, generally exhibit the following characteristics upon administration to the subject:

(i) a first phase, during which the plasma concentration of the isosorbide-5-mononitrate is maintained at a sub-therapeutic level in the blood stream of the subject for at least about 2 hours to about 12 hours following administration; followed by

(ii) a second phase, during which the plasma concentration of the ISMN in the blood stream of the subject is maintained above a minimum therapeutic level for about 6 to about 18 hours; optionally followed by

(iii) a third phase, during which the plasma concentration of the ISMN in the blood stream drops below the therapeutic level for about 1 to about 10 hours.

The therapeutic level is the minimum blood plasma concentration of ISMN that is therapeutically effective in the subject. One of skill in the art will recognize that the therapeutic level may vary depending on the individual being treated and the severity of the condition. For example, the age, body weight, and medical history of the individual subject may affect the therapeutic efficacy of the therapy. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual subject response. Typically, the minimum blood plasma concentration required to achieve a therapeutic effect using IS-5-MN is about 50 to about 200 ng/ml, about 50 to about 150 ng/ml, or any amount in between; for example, about 100 ng/ml. The minimum therapeutic plasma concentration for IS-2-MN, is about 10 to about 100 ng/ml, about 10 to about 50 ng/ml, or any amount in between; for example, about 20 ng/ml.

While lower plasma concentrations may not achieve a therapeutic effect, they are useful in the present invention for treating, preventing, reducing, reversing, and/or managing nitrate tolerance. For example, the formulations may provide sub-therapeutic levels of one or more ISMNs during the first and third phases for up to a total of 20 hours, including, for example, about 3 to about 20 hours, about 3 to about 16 hours, about 3 to about 12 hours, about 3 to about 10 hours, about 3 to about 6 hours, about 6 to about 20 hours, or about 6 to about 16 hours, about 6 to about 12 hours, about 6 to about 10 hours, or about 6 to about 8 hours, or any hour or fraction of time in between; the formulations may provide sub-therapeutic levels of the one or more ISMNs during the first and third phases for a total of about 3, 4, 5, 6, 7, 8, 9, 10, 11, 12, 13, 14, 15, 16, 17, 18, 19, or 20 hours, or any hour or fraction of time in between. These sub-therapeutic phases typically occur during a period of prolonged inactivity or minimum risk period for the subject, such as during sleep. This ensures that the lowest plasma levels of ISMN coincide with the period of least physical stress on the subject, as well as the period during which morning pathologies are least likely. In this manner, the ISMN formulations treat, prevent, reduce, reverse, and/or manage nitrate tolerance in subjects receiving such treatments.

The first phase provides for a delay in the release of therapeutic concentrations of one or more ISMNs. This permits the once-a-day formulation to treat morning pathologies. A subject can take the drug at night, prior to bedtime, but receive a therapeutically effective amount of one or more ISMNs by the early morning hours just prior to, and after, awakening. Accordingly, the first phase may delay the release of therapeutic concentrations of the one or more ISMNs for about 2 to about 12 hours, about 2 to about 10 hours, about 2 to about 8 hours or about 2 to about 6 hours, or any hour or fraction of time in between, following administration of the formulation; for example, the present formulations may delay release of therapeutic concentrations of the one or more ISMNs for about 2, 3, 4, 5, 6, 7, 8, 9, or 10 hours, or any hour or fraction of time in between, following administration.

During the second phase, the drug is released in an amount sufficient to exceed the minimum therapeutic level in the subject receiving the treatment. This therapeutic level is maintained for the length of time necessary to achieve the desired therapeutic outcome. Typically, the one or more ISMNs are maintained at or above the therapeutic level for about 6 to about 18 hours, about 6 to about 15 hours, about 6 to about 12 hours, about 8 to about 18 hours, about 8 to about 15 hours, about 8 to about 12 hours, about 8 to about 10 hours, about 10 to about 18 hours, about 10 to about 15 hours, or about 10 to about 12 hours, or any hour or fraction of time in between; accordingly, one or more ISMNs are maintained at or above the therapeutic level for about 6, 7, 8, 9, 10, 11, 12, 13, 14, 15, 16, 17, or 18 hours, or any hour or fraction of time in between, measured from the end of the first phase. In this manner, the present formulations extend release of one or more ISMNs to provide therapeutically effective amounts of nitrate throughout the day.

The washout period may be provided, all or in part, during the first phase. Alternatively, all or part of the washout period may be provided during the third phase. During the optional third phase, the plasma concentration of one or more ISMNs in the blood stream is permitted to drop below the therapeutic level for about 0 to about 10 hours, about 1 to about 8 hours, about 1 to about 6 hours, or from about 1 to about 4 hours, or any hour or fraction of time in between; alternatively, the one or more ISMNs in the blood stream is permitted to drop below the therapeutic level for about 0, 1, 2, 3, 4, 5, 6, 7, or 8 hours, or any hour or fraction of time in between.

As compared to the maximum ISMN levels during the second phase, the level to which the blood plasma concentration of ISMN falls during the washout period may exhibit a ratio (peak-to-trough) of from about 2:1 to about 10:1 or greater, and includes any whole number and/or fraction in between the listed ratios. Thus, the peak-to-trough ratio may be about 2:1, 3:1, 4:1, 5:1, 6:1, 7:1, 8:1, 9:1, 10:1, or greater.

In general, the total daily dosage of ISMN in the delayed onset, extended release formulations described herein is from about 10 mg to about 500 mg, about 10 mg to about 250 mg, about 10 mg to about 150 mg, or from about 30 mg to about 120 mg, or any whole number or fraction in between. A single dose may be formulated to contain about 1, 10, 15, 20, 25, 30, 35, 40, 45, 50, 55, 60, 70, 80, 90, 100, 120, 150, 200, 250, 300, 350, 400, 450, or 500 mg of one or more ISMNS. In one embodiment, a single dose contains 30, 60, 90, or 120 mg of one or more ISMNs.

In one embodiment, one or more ISMNs are provided in a delayed onset, extended release formulation suitable for once-daily oral administration that exhibits the pharmacokinetic profile described above. The delayed onset, extended release formulation provides a subject with therapeutic plasma levels of ISMN in the early morning hours and throughout the day, and also provides a washout phase to treat, prevent, reduce, reverse, and/or manage nitrate tolerance.

In one embodiment, suitable delayed onset, extended release formulations for use in the present methods typically comprise a core of one or more ISMNs, and/or pharmaceutically acceptable salts thereof, and optionally one or more pharmaceutically acceptable excipients to form an ISMN mixture.

In an instant or rapid release core, for example, the core may further comprise a polymeric material comprising a major proportion (i.e., greater than 50% of the total polymeric content) of one or more pharmaceutically acceptable water soluble polymers, and optionally a minor proportion (i.e., less than 50% of the total polymeric content) of one or more pharmaceutically acceptable water insoluble polymers.

In an extended release core, for example, the core may further comprise a polymeric material comprising a major proportion (i.e., greater than 50% of the total polymeric content) of one or more pharmaceutically acceptable water insoluble polymers, and optionally a minor proportion (i.e., less than 50% of the total polymeric content) of one or more pharmaceutically acceptable water soluble polymers.

The formulations may optionally contain a coating membrane partially or completely surrounding the core, comprising a major proportion of one or more pharmaceutically acceptable film-forming, water-insoluble polymers, and optionally a minor proportion of one or more pharmaceutically acceptable film-forming, water-soluble polymers.

The thickness of the coating membrane, the amount of polymer in the coating membrane and the core, and the ratio of water-soluble polymers to water-insoluble polymers in the coating membrane and core are generally selected such that the formulation initially delays the release of the ISMN, and then releases the ISMN from the formulation at a sustained rate for a specified period of time following oral administration, as described above. The rate of ISMN release typically exhibits a Tₘₐₓ from about 3 to about 12 hours, or any hour or fraction of time in between; and achieves a therapeutically effective concentration of ISMN for about 6 to about 18 hours, or any hour or fraction of time in between, during a 24 hour period of time.

The in vitro dissolution profile of the delayed onset, extended release ISMN formulations of the invention may correspond to the following:

(1) about 0 to about 10% of the one or more ISMNs are released between about 0 and about 2 hours;

(2) less than 50% is released after about 4 hours;

(3) greater than 50% is released after about 10 hours.

One of skill in the art is familiar with the techniques used to determine such dissolution profiles. The standard methodologies set forth in the U.S. Pharmacopeia, which is incorporated herein by reference in relevant part, may be used. For example, the dissolution profile may be measured in either a U.S. Pharmacopeia Type I Apparatus (baskets) or a U.S. Pharmacopeia Type II Apparatus (paddles). For pH-independent formulations, the formulations may be tested in phosphate buffer at pH 6.8, 37° C., and 50-100 rpm. For pH-dependent formulations, the formulations may be tested in 0.01-0.1 N HCl for the first 2 hours at 37° C. and 50-100 rpm, followed by transfer to phosphate buffer at pH 6.8 for the remainder of the test. Other buffer systems suitable for measuring the dissolution profile for pH-dependent and pH-independent formulations are well-known to those of skill in the art.

The dissolution profile of the present delayed onset, extended release ISMN formulations may substantially mimic one or more of the profiles provided below, based on in vivo release rates.

| | % Released | | |
|---|---|---|---|
| Time (hours) | Profile A | Profile B | Profile C |
| 0 | 0 | 0 | 0 |
| 5 | 0 | 0 | 0 |
| 6 | 50 | 25 | 10 |
| 7 | 75 | 44 | 19 |
| 8 | 88 | 58 | 27 |
| 9 | 94 | 68 | 34 |
| 10 | 97 | 76 | 41 |
| 11 | | 82 | 47 |
| 12 | 100 | 87 | 52 |
| 14 | | 92 | 62 |
| 16 | | 96 | 69 |
| 18 | | 98 | 75 |
| 20 | | 100 | 80 |

The formulations that can be used in the present methods may include any number of pharmaceutically acceptable excipients. Suitable excipients include, but are not limited to, carriers, such as sodium citrate and/or dicalcium phosphate; fillers and/or extenders, such as stearates, silicas, gypsum, starches, lactose, sucrose, glucose, mannitol, talc, and/or silicic acid; binders, such as hydroxymethyl-cellulose, alginates, gelatin, polyvinylpyrrolidone, sucrose and/or acacia; humectants, such as glycerol; disintegrating agents, such as agar, calcium carbonate, potato and/or tapioca starch, alginic acid, certain silicates, and/or sodium carbonate; solution retarding agents, such as paraffin; absorption accelerators, such as quaternary ammonium compounds; wetting agents, such as cetyl alcohol and/or glycerol monostearate; absorbents, such as kaolin and bentonite clay; lubricants, antiadherants, glidants, antisticking agents, and antitacking agents, such as talc, calcium stearate, magnesium stearate, aerosil (colloidal silicon dioxide), solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices.

Examples of suitable organic acids include, but are not limited to, adipic acid, ascorbic acid, citric acid, fumaric acid, malic acid, succinic acid, tartaric acid, and mixtures thereof. In some embodiments, the formulation includes an organic acid, and in others, an organic acid is excluded. Suitable organic bases, include, but are not limited to, sodium citrate, sodium succinate, sodium tartrate, potassium citrate, potassium tartrate, potassium succinate, and mixtures thereof. Suitable diluents include, but are not limited to, lactose, talc, microcrystalline cellulose, sorbitol, mannitol, xylitol, fumed silica, stearic acid, magnesium stearate, sodium stearate, and mixtures thereof. In some embodiments, the concentration of the diluent, for example talc or magnesium stearate, may be higher.

The core may also include additional pharmaceutically acceptable excipients including, but not limited to lubricants, dispersing agents, plasticizers, and surfactants. Suitable lubricants include, but are not limited to, talc, aerosil, and magnesium stearate. A suitable surfactant includes, but is not limited to, sodium lauryl sulfate.

The pharmaceutically acceptable excipients in the formulations may be included, for example, with the ISMN and/or the polymeric material in the core. Optionally, the excipients may be provided in the coating membrane. In one embodiment, the core contains a total of about 0% (w/w) to about 60% (w/w), or any percentage in between, of talc, magnesium stearate, and/or aerosil. In another embodiment, the polymeric material comprises a total of about 0% (w/w) to about 65% (w/w), or any percentage in between, of talc, magnesium stearate, and/or aerosil. In another embodiment, the coating membrane comprises a total of about 0% (w/w) to about 65% (w/w), or any percentage in between, of talc, magnesium stearate, and/or aerosil. For example, the core, polymeric material, and/or coating membrane may each comprise about 5, 10, 15, 20, 25, 30, 35, 40, 45, 50, or 60% (w/w), or any percentage in between, of talc, magnesium stearate, and/or aerosil.

Suitable plasticizers are selected based on the polymers used in the polymeric material. Suitable plasticizers include, but are not limited to, adipates, azelates, benzoates, citrates, isobucates, phthalates, sebacates, stearates, and glycols. For example, tributyl citrate is a suitable plasticizer for EUDRAGIT™ RS and EUDRAGIT™ RL; and dibutyl sebacate is a suitable plasticizer for cellulose acetate and cellulose acetate phthalate. The amount of plasticizer used in the polymeric solution/suspension may range from about 10% to about 50% relative to the weight of the dry polymer.

Water-soluble polymers include those which are freely water permeable and porous polymers. Water-insoluble polymers include those that are slightly water permeable or water impermeable and non-porous polymers. The polymeric material may substantially comprise a water insoluble polymer or a polymer that is slightly permeable to ISMN and water. Alternatively, the polymeric material may also include a minor proportion of a water soluble polymer and/or a polymer that is freely permeable to ISMN and water. The suitable ratio of water soluble to water insoluble polymer will vary depending on the particular polymers selected.

Suitable water soluble polymers include, but are not limited to, polyvinyl alcohol, polyvinylpyrrolidone, methyl cellulose, hydroxypropyl cellulose, hydroxypropyl methyl cellulose, polyethylene glycol, and/or mixtures thereof.

EUDRAGIT™ polymers (available from Rohm Pharma) are polymeric lacquer substances based on acrylates and/or methacrylates. EUDRAGIT™ RL and RS are acrylic resins comprising copolymers of acrylic and methacrylic acid esters with a low content of quaternary ammonium groups. The polymers swell in water and digestive juices, in a pH-independent manner. In the swollen state, they are permeable to water and to dissolved active compounds. The quaternary ammonium groups are present as salts and give rise to the permeability of the polymers.

A suitable polymer that is freely permeable to ISMNs and water includes the polymer EUDRAGIT™ RL. A suitable polymer which is only slightly permeable to water is EUDRAGIT™ RS. By combining these two polymers, or others exhibiting similar features, the release of ISMN from the formulation can be adjusted. In some methods, the ratio of EUDRAGIT™ RS: EUDRAGIT™ RL may be about 100:0, 90:10, 80:20, or 70:30, or any amount in between.

Suitable water insoluble polymers include, but are not limited to, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene), poly(propylene), poly(ethylene oxide), poly(ethylene terephthalate), poly(vinyl isobutyl ether), poly(vinyl acetate), poly(vinyl chloride), polyurethane, and/or mixtures thereof. Polymers which are slightly permeable to ISMN and water include, but are not limited to, EUDRAGIT™ L and EUDRAGIT™ RS. Other suitable polymers which are slightly permeable to ISMN and water, and exhibit a pH-dependent permeability include, but are not limited to, EUDRAGIT™ L, EUDRAGIT™ S, and EUDRAGIT™ E.

EUDRAGIT™ L is an anionic polymer synthesized from methacrylic acid and methacrylic acid methyl ester that is insoluble in acids and pure water. It becomes soluble in a neutral to weakly alkaline environment by forming salts with alkali compounds. The permeability of EUDRAGIT™ L is pH dependent. Above pH 5.0, the polymer becomes increasingly permeable.

In one embodiment, the water insoluble polymer is a high molecular weight ethyl cellulose, such as ETHOCEL™ Standard Premium 100 and/or ETHOCEL™ Medium 100 (Dow Chemical). The use of higher molecular weight material like the 100 designation material limits breakage during formulation. The numerical designations for ethylcellulose generally correspond to the viscosity of the product, with a higher numerical designation indicating a greater viscosity and higher molecular weight. The 100 designation corresponds to a viscosity of about 85-110 cp as measured in a 5% solution in an 80% toluene-20% ethanol solvent. The useful ethylcellulose designations are typically 7 and higher, corresponding to a viscosity of at least 6 cp. Viscosities of more than 40 cp (designation 45 or higher) are useful for crystals to be compressed into tablets. A useful water soluble polymer is KOLLIDON™. KOLLIDON™ is available from BASF as soluble and/or insoluble polyvinylpyrrolidones of various molecular weights and particle sizes. For example, KOLLIDON™ 30 provides medium molecular weight (Mw 44,000-54,000) polyvinylpyrrolidones.

In one embodiment, the polymer includes ethylcellulose and hydroxypropylcellulose. The weight ratio of ethylcellulose:hydroxypropylcellulose can range from about 3:1 to about 30:1, or about 5:1 to about 18:1; thus, the ratio may be about 5:1, 6:1, 7:1, 8:1, 9:1, 10:1, 11:1, 12:1, etc. By providing the proper balance of ethylcellulose to hydroxypropylcellulose a polymer can be formed which will remain intact in the stomach (and afterwards) but is permeable to gastric fluids, which dissolve and leach out the ISMN.

Suitable components (e.g., polymers, excipients, etc.) for use in the present delayed onset, extended release formulations, and methods of producing delayed onset or extended release formulations, are described, e.g., in U.S. Pat. No. 4,863,742, which is incorporated by reference for these purposes.

In one embodiment, the delayed onset, extended release formulations of the present invention comprising one or more ISMNs, optional excipients, and polymeric materials are built on a central inert core. The inert core may comprise a nonpareil seed of sugar and/or starch having an average diameter in the range of about 0.30-1.10 mm, about 0.40-0.90 mm, or about 0.75-0.81 mm. The seed may be coated in a conventional coating pan or, alternatively, using an automated system such as a CF granulator, a GLATT fluidized bed processor, an AEROMATIC, a modified ACCELA-COTA, or any other suitably automated coating equipment (FREUND, GLATT, AEROMATIC and ACCELA-COTA are all Trademarks).

The ISMN formulations described herein may be produced according to the following processes. Due to the danger of explosion in the handling and transport of ISMNs, the compounds are typically supplied by the manufacturer in a blend with an inert ingredient, such as lactose. The (ISMN):(inert ingredient) typically varies, depending on the manufacturer, from about 50:50 up to 100:0 (pure ISMN). Other suitable inert ingredients and ratios are known to those of skill in the art. The ISMN and optional excipients (e.g., binders, wetting agents, etc.) are blended to form a homogeneous mixture. The mixture is typically passed through a No. 25-400 or 25-500 mesh screen using a milling machine to screen out agglomerates. Alternatively, the optional excipients can be blended together and milled, with the resulting mixture being blended together with the ISMN. Optionally, the milling process, with the optional excipients and active ingredient, can be carried out in a suitable media (organic or aqueous). Then, the liquid form of the drug may be applied to the nonpareil seeds.

The ISMN mixture is then applied to an inert core particle, such as a nonpareil seed. Alternatively, the ISMN and optional excipients can be provided in a solution or suspension, and then applied to the core particle. Typical core particles, such as seeds, may have a diameter in the range of about 0.30 mm to about 1.10 mm. The ISMN mixture may be applied using any suitable apparatus, such as a fluidized bed coater and/or a pan coating system.

A polymeric material, provided in a solution/suspension, can also be applied to the seeds. The ISMN mixture may be applied at the same time as the polymeric solution/suspension. Alternatively, the polymer solution/suspension may be applied after the ISMN has been applied. For example, the seeds may be coated with the ISMN mixture, dried, and then coated with the polymer solution/suspension. Optionally, the ISMN and polymer may be applied in an alternating manner. The polymer and ISMN, whether applied separately or together, can be formulated to provide active coated cores having the desired thickness and properties.

The solution/suspension of polymer typically comprises one or more polymers dissolved and/or suspended in a suitable solvent or mixture of solvents. Such polymers may comprise one or more pharmaceutically acceptable water-insoluble polymers, and optionally, a minor proportion of one or more pharmaceutically acceptable water-soluble polymers, or vice versa, depending on the desired role of the polymeric material. Suitable polymers are described above. The ratio of water insoluble to water soluble polymers is determined by the inherent solubility characteristics of the polymers selected. The solvent may be organic and/or aqueous. The concentration of the polymeric material in the solution/suspension is typically determined by the viscosity of the final solution. A suitable plasticizer, as described previously, may optionally be added to the polymer solution/suspension.

Suitable polymer solutions/suspensions include, but are not limited to:

a. 1%-10% polyvinylpyrrolidone in isopropanol or ethanol;

b. 5%-10% ethylcellulose in isopropanol;

c. 5%-10% hydroxypropylmethyl cellulose in methanol/methylene chloride 60/40;

d. 5% EUDRAGIT™ RL in isopropanol/acetone 60/40;

e. 5% EUDRAGIT™ RS in isopropanol/acetone 60/40;

f. 30% EUDRAGIT™ RS water dispersion;

g. 30% EUDRAGIT™ RL water dispersion;

h. 6% ETHOCEL™ 7 cps (ethylcellulose): KOLLIDON 30 (polyvinylpyrrolidone) (95:5) in isopropanol;

i. 5%-7% ETHOCEL™ 7 cps (ethylcellulose) in isopropanol

j. ETHOCEL™:polyvinylpyrrolidone in solution at 40:60, 50:50, 65:35, 70:30, 75:25, 80:20, 85:15, 90:10, 95:5, or any ratio in between.

After completing the formation of the polymer-coated cores, they are dried in a conventional drying oven at a suitable temperature, for example, about 35-65° C., or 40-60° C., or any temperature in between. Alternatively, other types of conventional pharmaceutical drying equipment can be used, such as fluid bed, vacuum, or microwave.

Additional components, such as additional polymeric coatings, may optionally be included in the formulation and applied to the polymer-coated core. Such polymers may comprise additional pharmaceutically acceptable, water-insoluble polymers, and optionally a minor proportion of one or more pharmaceutically acceptable film-forming, water-soluble polymers, as described above. Once such additional polymers have been applied, the resulting cores are typically dried again, as described above.

Additionally, one or more sealants and/or barriers can be applied to the formulation. Sealants and/or barriers are typically polymeric coatings applied to the outer surface of the formulation. For example, a sealant or barrier may function as an enteric coating so that the formulation is able to pass through the acidic environment of the stomach, to prevent agglomeration of the polymer-coated cores, or to protect or stabilize the dosage form prior to administration. Suitable sealants and barriers can be selected from any of the polymeric components described previously, including, for example, hydroxypropyl methylcellulose, hydroxypropyl cellulose, hydroxypropyl ethylcellulose, and xanthan gum. Such coats are also useful, for example, to prevent or minimize moisture uptake. Suitable sealant coats include, but are not limited to, acetates and other commercially available products known to those of skill in the art, such as OPADRY™ AMB (Colorcon Ltd.). The sealant coat may comprise any of the above mentioned pharmaceutically acceptable excipients.

The polymer-coated cores may then be formulated into a suitable dosage form. The compositions described above may be provided in any pharmaceutically acceptable dosage form, including, but not limited to, caplets, capsules, multi-particle suspensions, sachets, tablets, and/or minitablets. The minitablets may also be encapsulated, for example, into hard gelatin capsules.

The desired release rate may be obtained by providing a formulation containing polymer-coated cores that each exhibit a uniform rate of release. Alternatively, the desired release rate may be obtained by providing a formulation containing polymer-coated cores that separately exhibit different rates of release, but together achieve the desired overall rate of release for the formulation.

Any of the pharmaceutical compositions described above may further comprise one or more pharmaceutically active compounds other than ISMN. Such compounds may be provided to treat the same condition being treated with ISMN, or a different one. Those of skill in the art are familiar with examples of the techniques for incorporating additional active ingredients into the delayed onset, extended release formulations comprising ISMN. Alternatively, such additional pharmaceutical compounds may be provided in a separate formulation and co-administered to a subject with an ISMN composition. Such separate formulations may be administered before, after, or simultaneously with the administration of the ISMN.

While not wishing to be bound by any particular theory, it is believed that formulations of the present invention are uniquely suited for nighttime administration. As will be demonstrated in the Examples below, conventional formulations of ISMN, and tablets in particular, if not appropriately designed in accordance with the present invention, exhibit a delayed transit while passing through the stomach and into the intestine. This delay in transit increases during sleeping hours, when gastrointestinal motility is considerably slowed, resulting in a considerable increase in the duration of exposure of the body to bioavailable ISMN. As a result of this gastrointestinal transit effect, many formulations designed for morning administration may not be suitable for nighttime administration. For example, a tablet formulation designed for morning administration may produce twice the level of ISMN in the body when administered at night, which is clearly undesirable. The present invention solves this problem through the use of, for example, multiparticulate formulations that are designed to minimize the presently observed gastrointestinal transit effect found with other ISMN formulations.

Additionally, the discovery of this presently observed gastrointestinal transit effect has led to formulations of the present invention that use less ISMN to achieve a therapeutic effect that is equivalent to that achieved by higher doses of ISMN in conventional formulations. For example, methods and formulations are provided in which a tablet ISMN formulation is administered, wherein the formulation includes an amount of ISMN that is less than that necessary to produce a therapeutic effect if the formulation were administered in the morning. But when administered at night these formulations produce a therapeutically effective blood concentration.

Additionally, the present invention provides methods of increasing the bioavailability of an ISMN tablet formulation. Such methods involve informing a subject taking a tablet formulation of ISMN, designed for morning administration, to administer the tablet formulation in the evening before bed. Such methods would be desirable to improve the bioavailability to subjects in need of such improvement.

The invention is further illustrated by reference to the following examples. It will be apparent to those skilled in the art that many modifications, both to materials and methods, may be practiced without departing from the purpose and scope of the invention.

### EXAMPLES

### Example 1

### Preparation of Sustained Release ISMN Tablets

A sustained release ISMN tablet was prepared as follows:

| Ingredient | Mg/Tablet |
|---|---|
| IS-5-MN/Lactose (80:20) | 60.00 |
| (IS-5-MN equivalent) | |
| METHOCEL ™ K100M | 240.00 |
| Premium 2208 | |
| Avicel pH101 | 82.6 |
| Aerosil | 1.6 |
| Colloidal Silicon Dioxide | |
| Magnesium Stearate | 0.8 |

The colloidal silicon dioxide and METHOCEL™ were sieved together through a 0.5 mm sieve. All excipients (except the magnesium stearate) were placed in a blender and mixed for 10 minutes. The magnesium stearate was added and the mixture was mixed for an additional 5 minutes. The blend was compressed into tablets on a rotary tablet machine. Where necessary, coating was performed by placing the tablets in a coating machine (Accelacota) and spraying them with a solution/suspension of EUDRAGIT™ L until the required weight is achieved (e.g., about 5%-50% weight gain). This sustained release tablet formulation was designed to provide a similar pharmacokinetic profile to the commercially available ISMN tablet, IMDUR™, a product indicated for morning administration.

### Example 2

### Pharmacokinetics of Tablets Administered in AM and PM

A balanced, randomized, crossover study was conducted to assess the pharmacokinetics of various coated and uncoated IS-5-MN tablets when administered at night. The study investigated the following IS-5-MN formulations:

60 mg SR uncoated (SR), and

60 mg SR EUDRAGIT™ L coated (L-SR).

Fifteen subjects participated in the study. Subjects were administered a dosage form of IS-5-MN at about 10 P.M. in each of five treatment periods.

A second balanced, randomized, crossover study was conducted to assess the pharmacokinetics of 60 mg uncoated IS-5-MN tablets when administered in the morning. Approximately twenty-four subjects participated in this study. Subjects were administered a dose of the 60 mg uncoated IS-5-MN formulation at about 8 A.M. (SR-AM) in each of two treatment periods. Results were compared with the 60 mg SR uncoated formulation administered at night (described above).

These two studies were conducted on different days, testing different populations of subjects, and using drug formulations from different batches. Thus, some variation is to be expected in making comparisons between the studies.

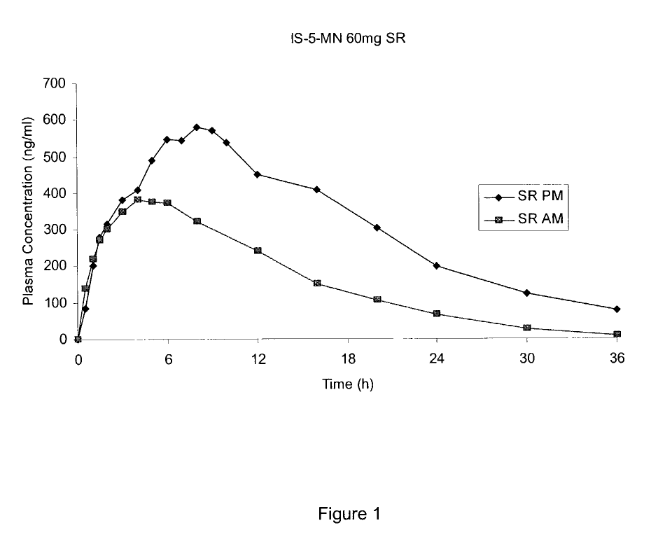

FIG. 1 illustrates the results observed when a sustained release tablet formulation was administered in the morning (at about 8 AM), or at night (at about 10 PM). Table 1 shows Cmax, AUC₍₀₋ₜ₎, and AUC_{(inf.)} for these results.

TABLE 1

| Measurement | SR-AM | SR-PM | Fold Difference |
|---|---|---|---|
| Cmax | 409.25 | 682.81 | 1.67 |
| AUC₍₀₋ₜ₎ | 5669.98 | 10913.35 | 1.93 |
| AUC_{(inf.)} | 5732.691 | 12036.63 | 2.10 |

As the data show, administering a sustained release ISMN tablet formulation at night, when that formulation has been designed for morning administration, can lead to unwanted consequences, such as significant increases in maximum plasma concentration and bioavailability. Moreover, because of the extended effect (note the plasma concentration at 24 hours and later), this product would not produce a desired washout period. Thus, it is not sufficient to simply administer an existing formulation, which is designed for morning administration, at night. Unless modified to account for the effects observed herein, such formulations would not be acceptable for nighttime administration.

### Example 3

### Pharmacokinetics of Coated and Uncoated SR Tablets

Example 2 above demonstrates how formulations that are not specifically designed for nighttime administration may not be appropriate for administration in that manner. This Example extends the conclusions drawn in Example 2 and shows how simple modifications, made without an appreciation of the GI transit phenomenon observed herein, can cause additional undesirable effects.

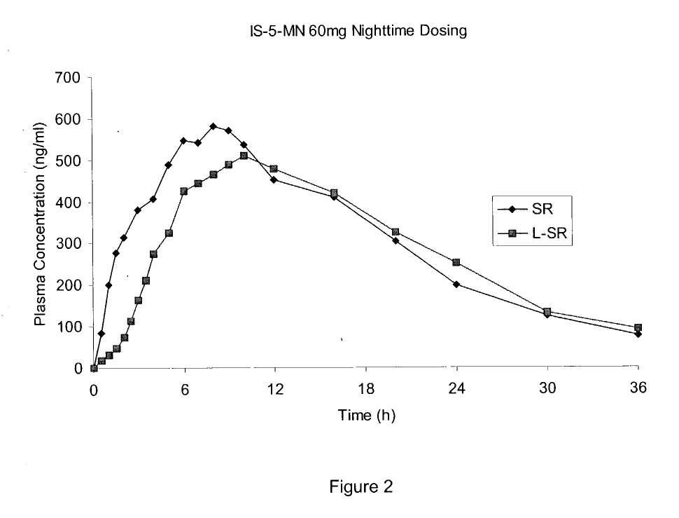

FIG. 2 shows the results observed when a sustained release tablet, prepared as described in Example 1 above and tested in Example 2 above, is administered at night, in either coated or uncoated form. The uncoated tablet is identical to that described in the previous example, and the coated tablet has been coated to a 15% weight gain with EUDRAGIT™ L, an enteric coating polymer that preferentially dissolves at a pH higher than about 5.5. Thus, the coated form would not dissolve until after exiting the acidic stomach. The uncoated sustained release formulation, on the other hand, would begin releasing its contents upon dissolution in the stomach. The results are summarized in Table 2.

TABLE 2

| Measurement | Uncoated SR-PM | Coated SR-PM |
|---|---|---|
| Cmax | 682.81 | 595.86 |
| AUC₍₀₋ₜ₎ | 10913.35 | 10156.63 |
| AUC_{(inf.)} | 12036.25 | 11282.18 |
| Tmax | 8.2 | 9.5 |

As expected, in comparing the uncoated and coated, the uncoated formulation appears in the plasma earlier, achieving therapeutic levels less than one hour after administration. The coated formulation required over two hours to achieve the same level. The uncoated formulation exhibited a slightly higher area under the curve (AUC) and maximum plasma concentration (Cmax), both of which effects are likely due to the slower nighttime GI transit and increased exposure of the dissolved drug to the GI tract compared to the delayed release. The later initial release due to the delayed release also had the effect of shifting the entire plasma concentration curve to the right, resulting in even higher levels of drug in the blood after twenty-four hours. The high plasma concentration at twenty-four hours makes this coated tablet a poor candidate for achieving a washout.

Thus, because of the nighttime GI transit phenomenon described herein, the uncoated SR tablet produces very high pharmacokinetic parameters, resulting in a prolonged plasma concentration, making the formulation undesirable for repeated daily administration where a washout is necessary. Coating the SR tablet with an enteric coating even further enhances the problem, and produces very prolonged plasma ISMN concentrations. Thus, unless modified to account for the effects observed herein, these formulations would not be acceptable for nighttime administration.

### Example 4

### Production of IS-5-MN-Loaded Cores

In the first step of production, an IS-5-MN mixture (80:20 on lactose and 5% Aerosil) was prepared. The IS-5-MN powder, lactose, aerosil, talc, and fumaric acid (where applicable), were bag blended for five minutes. This IS-5-MN mixture was then applied, along with a binder solution comprising polyvinylpyrrolidone (4% KOLLIDON™ 30 in isopropanol), to nonpareil seeds to produce IS-5-MN loaded cores. Several formulations of IS-5-MN-loaded cores were produced. The particular components used to make the cores are described in Table 3.

TABLE 3
Compositions of IS-5-MN (60 mg) Loaded Cores

| | Loaded Cores | Loaded Cores | Loaded Cores |
|---|---|---|---|
| | for PD15497 | for PD15498 | for PD15499 |
| Compound | concentration (mg/g) | | |
| IS-5-MN (80:20 on | 474.6 | 407.1 | 474.1 |
| lactose and | | | |
| 5% Aerosil) | | | |
| Talc | 150.4 | 55.6 | 150.2 |
| Nonpareil seeds | 360.6 | 355.9 | 360.2 |
| (0.71-0.85 mm) | | | |
| Milled fumaric acid | — | 154.1 | — |
| 4% KOLLIDON ™ 30 | 14.42 | 27.4 | 15.5 |
| Solution in | | | |
| isopropanol | | | |

The IS-5-MN loaded cores were then oven dried at 50° C. for 20 hours to remove solvent. The dried cores were passed through a sieve to remove agglomerates. The resulting drug loaded cores were analyzed as described below.

The IS-5-MN-loaded cores formed from the above-described components were evaluated for particle size. Results are reported in Table 4.

TABLE 4
Particle size analysis of IS-5-MN (60 mg) Loaded Cores

| | Percent Retained | | |
|---|---|---|---|
| | Loaded Cores | Loaded Cores | Loaded Cores |
| Size (microns) | for PD15497 | for PD15498 | for PD15499 |
| <500 | 0 | 0 | 0 |
| 500 | 0 | 0 | 0 |
| 710 | 0 | 0 | 0 |
| 850 | 50.0 | 39.2 | 46.0 |
| 1000 | 48.0 | 56.9 | 42.0 |
| 1180 | 2.0 | 3.9 | 12.0 |
| 1400 | 0 | 0 | 0 |
| 1700 | 0 | 0 | 0 |

The dissolution characteristics of the IS-5-MN loaded cores were also evaluated. The cores were tested in 0.05 M phosphate buffer (pH 6.8) using an USP Type II apparatus at 50 rpm (37° C.±0.5° C.). Results are reported in Table 5.

TABLE 5
Dissolution Profile of IS-5-MN (60 mg) Loaded Cores

| | Loaded Cores | Loaded Cores | Loaded Cores |
|---|---|---|---|
| | for PD15497 | for PD15498 | for PD15499 |
| Time (minutes) | Percent Released | | |
| 0 | 0 | 0 | 0 |
| 15 | 88.7 | 97.6 | 97.2 |
| 30 | 94.1 | 100.7 | 99.1 |
| 45 | 96.5 | 101.6 | 99.9 |
| 60 | 98.0 | 101.9 | 100.7 |

Finally, the loaded cores were evaluated for potency and moisture content, which total water content of the formulation, as determined by the Karl Fischer method. Results are reported in Table 6. The term “potency” in this example measures the amount of IS-5-MN that is present. The “theoretical potency” refers to the amount of IS-5-MN that was actually applied to the cores. The actual potency refers to the amount of IS-5-MN that is actually present in the loaded core, as measured by HPLC analysis. The difference between the two values, expressed as a percentage ([Actual Potency]/[Theoretical Potency]) and termed the “percent label claim,” takes into account any IS-5-MN that was lost during processing. The percentage is expressed as the w/w of the IS-5-MN measured.

TABLE 6
Potency and Moisture Content of IS-5-MN (60 mg) Loaded Cores

| | Loaded Cores | Loaded Cores | Loaded Cores |
|---|---|---|---|
| Measurement | for PD15497 | for PD15498 | for PD15499 |
| Theoretical Potency | 361.0 | 298.0 | 360.0 |
| (mg/g) | | | |
| Actual Potency (mg/g) | 354.5 | 304.9 | 351.6 |
| % Label Claim | 98.2 | 98.7 | 97.7 |
| % Moisture Content | 0.6534 | 0.8535 | 0.6509 |

### Example 5

### Production of Polymer-Coated Cores

In the next stage of production, the drug-loaded cores were coated with a polymer solution. Talc was applied at the same time as the polymer solution to prevent agglomeration of the cores. The polymeric solution was coated onto the loaded cores at a rate of about 9 g/min. The talc was applied at a rate of about 2.5 g/min. The compositions used to formulate the polymer-coated cores are described in Table 7.

TABLE 7
Compositions for Formulatin IS-5-MN (60 mg) Polymer-Coated Cores

| | Polymer- | Polymer- | Polymer- |
|---|---|---|---|
| | Coated Cores | Coated Cores | Coated Cores |
| | for PD15497 | for PD15498 | for PD15499 |
| Compound | Concentration (Kg) | | |
| IS-5-MN-loaded cores | 1.500 | 1.500 | 1.500 |
| Talc | 0.734 | 0.573 | 0.557 |
| 6% Coating Solution | 2.754 | 2.005 | 2.014 |
| ETHOCEL ™ 7 cps: | | | |
| KOLLIDON ™ 30 | | | |
| Solution in | | | |
| isopropanol (95:5) | | | |

After coating, the polymer-coated cores were oven dried at 50° C. for 20 hours. The dried cores were passed through a sieve to remove agglomerates. The resulting polymer-coated cores were analyzed as described below.

The dissolution characteristics of the IS-5-MN polymer-coated cores were evaluated. The cores were tested in 0.05 M phosphate buffer (pH 6.8) using an USP Type II apparatus at 50 rpm (37° C.±0.5° C.). Results are reported in Table 8.

TABLE 8
Dissolution Profile of IS-5-MN (60 mg) Polymer-Coated Cores

| | Polymer- | Polymer- | Polymer- |
|---|---|---|---|
| | Coated Cores | Coated Cores | Coated Cores |
| | for PD15497 | for PD15498 | for PD15499 |
| Time (hours) | Percent Released | | |
| 0 | 0 | 0 | 0 |
| 1 | 0 | 0 | 0 |
| 2 | 0 | 1.4 | 4.5 |
| 3 | 0 | 6.5 | 4.4 |
| 4 | 3.0 | 12.6 | 14.5 |
| 6 | 10.0 | 27.0 | 46.8 |
| 8 | 22.1 | 41.9 | 72.4 |
| 12 | 56.7 | 68.2 | 91.9 |
| 24 | 101.4 | 93.6 | 97.9 |

The polymer-coated cores also were evaluated for potency and moisture content. Results are reported in Table 9. Moisture content was determined according to standard methodology set forth in the U.S. Pharmacopeia. The theoretical and actual potencies and the percent label claim are described in Example 4, except that IS-5-MN (60 mg) polymer-coated cores were tested in this example instead of the IS-5-MN (60 mg) loaded cores.

TABLE 9
Potency and Moisture Content of IS-5-MN
(60 mg) Polymer-Coated Cores

| | Polymer- | Polymer- | Polymer- |
|---|---|---|---|
| | Coated Cores | Coated Cores | Coated Cores |
| Measurement | for PD15497 | for PD15498 | for PD15499 |
| Theoretical Potency | 225.6 | 211.5 | 248.2 |
| (mg/g) | | | |
| Actual Potency (mg/g) | 230.9 | 218.0 | 269.1 |
| % Label Claim | 103.0 | 103.1 | 108.4 |
| % Moisture Content | 0.3273 | 0.2506 | 0.5392 |

### Example 6

### Production of IS-5-MN Oral Dosage Form

In the final stage of production, the polymer-coated cores were encapsulated to produce an oral dosage form. The polymer-coated cores were filled into Size 1, white opaque capsules using the Bosch GKF400S Encapsulating Machine. The final compositions, taking into account the amounts and proportions of all ingredients added at each stage of production, are provided in Table 10A.

TABLE 10A
Composition of IS-5-MN Oral Dosage Forms

| Ingredient | mg/g | mg/g | mg/g | Grade | Function |
|---|---|---|---|---|---|
| Batch | PD | PD | PD | | |
| Number | 15497 | 15498 | 15499 | | |
| Isosorbide 5 | 225.47 | 211.58 | 248.10 | EP | Active |
| Mononitrate | | | | | |
| Lactose | 56.39 | 52.90 | 62.0 | USP/EP | Diluent |
| Non Pareil | 225.47 | 243.50 | 248.10 | USP/EP | Inert Carrier |
| Seeds | | | | | |
| Aerosil 200 | 14.92 | 13.68 | 16.40 | USP/EP | Glidant |
| (Colloidal | | | | | |
| Silicon | | | | | |
| Dioxide | | | | | |
| Fumaric acid | | 105.34 | | | |
| Talc | 399.95 | 299.14 | 359.10 | USP/EP | Anti-Adherent |
| Kollidon | 12.46 | 21.43 | 13.60 | USP/EP | Binder/Controlled |
| (PVP) | | | | | release polymer |
| Ethocel | 65.40 | 51.98 | 52.80 | USP/EP | Controlled |
| | | | | | release Polymer |

The oral dosage form was evaluated as reported in Tables 10B and 11, below.

TABLE 10B
Analytical Results for IS-5-MN Oral Dosage Form

| Measurement | PD15497 | PD15498 | PD15499 |
|---|---|---|---|
| Formulation Details | 1.2% KOLLIDON ™ 30 | 4.6% KOLLIDON ™ 30 | 1.4% KOLLIDON ™ 30 |
| | 6.6% ETHOCEL ™ 7 cps | 5.2% ETHOCEL ™ 7 cps | 5.3% ETHOCEL ™ 7 cps |
| | | 10.1% Fumaric acid | |
| Mean Content Weight (g) | 0.25761 | 0.27714 | 0.22378 |
| Range (%) | 97.3-102.7 | 98.1-102.2 | 97.2-101.9 |
| Assay (mg/capsule) | 59.0 | 60.0 | 58.2 |
| % Label Claim (60 mg) | 98.3 | 100.0 | 96.9 |
| Solvent Residue (%) | 0.3 | 0.03 | 0.03 |
| Mean Dose (mg/capsule) | 59.1 | 60.6 | 557.1 |
| Range (% of label claim) | 95.0-100.8 | 99.2-103.5 | 93.2-97.5 |
| CV (%) | 1.9 | 1.5 | 1.5 |
| Related Substances | None Detected | None Detected | None Detected |
| Moisture Content (%) | 0.9524 | 0.9682 | 1.0729 |

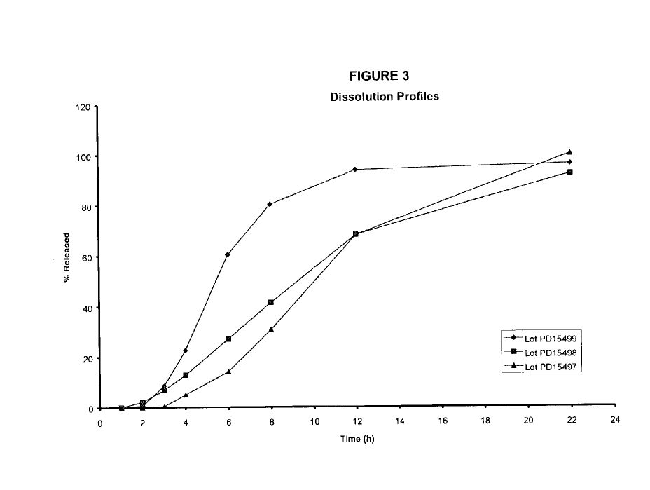

The dissolution characteristics of the IS-5-MN oral dosage forms were evaluated. The dosage forms were tested in 0.05 M phosphate buffer (pH 6.8) using an USP Type II apparatus at 50 rpm (37° C.±0.5° C.). Results are reported in Table 11 and illustrated in FIG. 3.

TABLE 11
Dissolution Profile of IS-5-MN (60 mg) Oral Dosage Form

| | PD15497 | PD15498 | PD15499 |
|---|---|---|---|
| Time (hours) | Percent Released | | |
| 0 | 0 | 0 | 0 |
| 1 | 0 | 0.1 | 0 |
| 2 | 0 | 2.1 | 0.7 |
| 3 | 0.4 | 6.9 | 8.6 |
| 4 | 5.0 | 12.9 | 22.7 |
| 6 | 14.2 | 27.1 | 60.5 |
| 8 | 30.8 | 41.5 | 80.4 |
| 12 | 68.2 | 68.3 | 94.0 |
| 24 | 100.3 | 92.4 | 96.3 |

### Example 7

### Biostudy of IS-5-MN Oral Dosage Form

balanced, randomized, crossover study was conducted to assess the bioavailability of IS-5-MN following administration of the IS-5-MN oral dosage form to a subject. The study compared the bioavailability of 3 different 60 mg IS-5-MN formulations and 60 mg of IMDUR™ (Key Pharmaceuticals), when dosed at night. Twelve subjects enrolled and completed the study. Subjects were administered a dosage form of IS-5-MN at night in each of four treatment periods, with a seven day washout between treatment periods. Plasma samples were taken from the test subjects at 0, 1, 2, 4, 4.5, 5, 5.5, 6, 7, 8, 9, 10, 11, 12, 16, 20, 24, 30, and 36 hours following administration of the dosage form. Plasma concentrations of the IS-5-MN were measured using GC with electron capture detection. The calibration range was 10-1000 ng/ml.

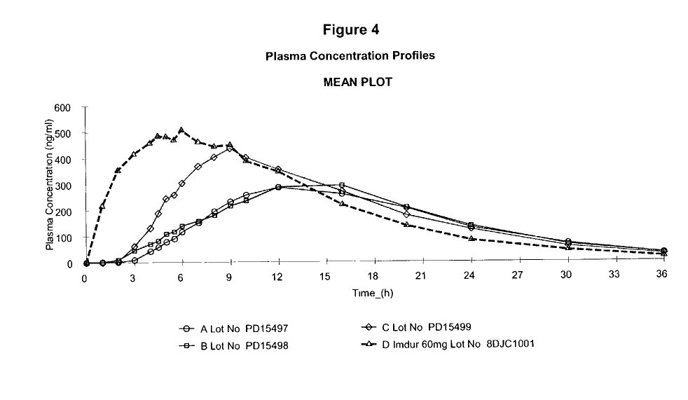

FIG. 4 illustrates the blood plasma concentration of IS-5-MN following administration of different formulations of IS-5-MN (60 mg) dosage forms and IMDUR™ (60 mg) at night. As seen in this figure, the IS-5-MN formulations provided an initial delay phase, where concentrations of the nitrate were below the therapeutic level. This was followed by an extended therapeutic phase, where the nitrate was maintained above the therapeutic level, followed by a washout phase.

Additional parameters measured included area under the plasma concentration curve extrapolated to infinity (AUC_{inf}) and up to the last sampling (AUCₐₗₗ); the maximum plasma concentration of the drug (Cₘₐₓ) and the time of its occurrence (tₘₐₓ); the bioavailability (Fᵣₑₗ) of the test formulations relative to that of the reference; the time required for the drug plasma concentration to decrease by 50% (t_{1/2}); and the terminal first order elimination rate constant, lambda z. T_{lag} is the time prior to the first quantifiable concentration. The raw data is summarized in Tables 12 and 13. The data relative to IMDUR™ (60 mg) is summarized in Table 14.

TABLE 12
Biostudy Data (log₁₀ -transformed

| | PD15497 | PD15498 | PD15499 | IMDUR ™ |
|---|---|---|---|---|
| Parameter* | Mean (gsd) | | | |
| AUC_{inf} | 5291.61 | 5451.04 | 6593.55 (1.17) | 7642.29 (1.12) |
| | (1.19) | (1.27) | | |
| 90% Cl | 63-78 | 65-80 | 79-98 | — |
| Cₘₐₓ | 294.66 | 303.23 | 442.68 (1.32) | 528.28 (1.13) |
| | (1.21) | (1.29) | | |
| 90% Cl | 49-65 | 50-67 | 74-98 | — |
| AUCₐₗₗ | 5017.95 | 5178.60 | 6372.15 (0.07) | 7493.06 (0.05) |
| | (0.08) | (0.11) | | |
| 90% Cl | 61-75 | 62-78 | 77-97 | — |

90% Cl indicates the 90% confidence intervals relative to IMDUR ™.

TABLE 13
Biostudy Data (non-transformed)

| | | | | |
|---|---|---|---|---|
| Para- | PD15497 | PD15498 | PD15499 | IMDUR ™ |

| meter* | Mean ± S.D. | | | |
|---|---|---|---|---|
| AUC_{inf} | 5360.82 ± | 5581.05 ± | 6666.66 ± | 7688.98 ± |
| (ng/ml/h) | 879.03 | 1174.41 | 1031.81 | 869.25 |
| AUCₐₗₗ | 5086.20 ± | 5307.77 ± | 6448.60 ± | 7536.90 ± |
| (ng/ml/h) | 850.98 | 1137.32 | 1028.96 | 836.73 |
| Cₘₐₓ | 299.44 ± | 311.82 ± | 457.83 ± | 531.81 ± |
| (ng/ml) | 54.20 | 72.36 | 117.98 | 65.54 |
| tₘₐₓ | 12.58 ± | 14.00 ± | 10.00 ± | 5.63 ± 1.33 |
| (h) | 2.28 | 2.09 | 2.3 | |
| Lambda | 0.12 ± | 0.12 ± 0.02 | 0.12 ± 0.01 | 0.13 ± 0.02 |
| z (h⁻¹) | 0.01 | | | |
| T_{1/2} | 6.00 ± | 5.95 ± 1.17 | 5.65 ± 0.57 | 5.60 ± 0.72 |
| (h⁻¹) | 0.49 | | | |
| Fᵣₑₗ (%) | 69.88 ± | 73.05 ± | 88.06 ± | — |
| | 10.10 | 14.02 | 19.11 | |
| T_{lag} (h) | 2.25 ± 0.87 | 1.33 ± 0.65 | 1.58 ± 0.79 | 0 |
| range | (0-3) | (0-2) | (0-3) | (0) |

Fᵣₑₗ (%) refers to the bioavailability of the test compound relative to IMDUR™. A test compound exhibiting a Fᵣₑₗ (%) that is greater than about 65-70% (relative to IMDUR™) indicates good bioavailability of the IS-5-MN. Rel Cₘₐₓ (%) refers to the peak concentration of the test compound relative to IMDUR™. A high Rel Cₘₐₓ (%) indicates that the test compound exhibits a peak to trough ratio that is similar to IMDUR™. The peak-to-trough ratio for IMDUR™ is about 5:1. A lower Rel Cₘₐₓ (%) suggests that a compound has a lower peak-to-trough ratio than IMDUR™. A higher Rel Cₘₐₓ (%) may allow the compound to achieve a therapeutically effective concentration of ISMN, while allowing sufficient time for a washout. T_{diff} refers the difference in tₘₐₓ of the test compound relative to IMDUR™. This reflects the difference in the delayed onset of release between IMDUR™ and the test compounds, which were designed to extend the delay period. As the results indicate, all of the test compounds achieved a longer delayed onset of release relative to IMDUR™.

TABLE 14
Biostudy Results Relative to IMDUR ™

| Relative PK Parameter* | PD15497 | PD15498 | PD15499 |
|---|---|---|---|
| Fᵣₑᵢ (%) | 70.0 | 73.1 | 88.1 |
| Rel. Cₘₐₓ (%) | 56.4 | 59.4 | 87.3 |
| +T_{diff} (h)** | +7.0 | +8.4 | +4.4 |

*Reference to IMDUR ™ IS-5-MN 60 mg formulation (Key Pharmaceuticals)
**T_{diff} = Tₘₐₓ of test − Tₘₐₓ of reference compound

### Example 8

### Use of Delayed Onset, Extended Release IS-5-MN Oral Dosage Form to Treat a Subject Suffering from Angina

A subject suffering from angina will receive a therapeutic benefit from the vascular relaxant effects of IS-5-MN and especially from the delayed onset, extended release oral dosage form described herein. Particular benefits include the avoidance and/or reduction in symptoms of angina at or around the time of awakening in the morning, and the continuance of this relief during the waking day, through periods of activity that would cause pain and discomfort in untreated subjects. The delayed onset, extended release oral dosage form is taken in the evening at bedtime. The delay in onset and subsequent release of IS-5-MN from the formulation ensures that therapeutic concentrations of drug are achieved prior to the subject awakening, thus protecting the subject from angina attacks in the high risk early morning period. The extended release from the formulation ensures that the subject is also protected throughout the high activity waking hours of the day. The treating physician will recognize the need to modify the dose according to the severity and frequency of symptoms. The recommended starting dose is 30 mg or 60 mg, once-daily. At the judgment of the treating physician, the dose may be increased to 120 mg, once daily, after several days. Rarely, 240 mg daily may be required.

### Example 9

### Use of Delayed Onset, Extended Release IS-5-MN Oral Dosage Form to Treat, Prevent, Reduce, Reverse, and/or Manage Nitrate Tolerance

A subject who requires constant treatment with IS-5-MN or other nitrates for the management of angina generally develops a tolerance to the effects of the medication. This is reflected in lack of anti-anginal effect, the occurrence of pain and discomfort, and restriction of activity, even in the presence of continuing therapy and increased dosages. This tolerance may be treated, prevented, reduced, reversed, and/or managed by the use of the present delayed onset, extended release IS-5-MN oral dosage form described herein. The product is taken in the evening at bedtime. The delay in onset coupled with the tapering of release at the end of the dosing interval ensures that the subject receives therapeutic amounts of ISMN in the morning and throughout the day, but also has a sufficiently long interval during which amounts of nitrate in the body fall below the therapeutic level so that tolerance does not develop, i.e., a washout period. The drug free period coincides with the lowest risk period for angina attacks (nighttime and during sleep) for the safety and comfort of the subject. The treating physician will recognize the need to modify the dose according to the severity and frequency of symptoms. The recommended starting dose is 30 mg or 60 mg, once-daily. At the judgment of the treating physician the dose may be increased to 120 mg, once daily, after several days. Rarely, 240 mg daily may be required.

### Example 10

### Use of Delayed Onset, Extended Release IS-5-MN Oral Dosage Form to Reverse Nitrate Tolerance

A subject who requires constant treatment with IS-5-MN or other nitrates for the management of angina develops tolerance to the effects of the medication. This occurs when a sufficient period has not been provided during which amounts of nitrate in the body fall below a threshold therapeutic level. The resulting development of tolerance is indicated by a lack of anti-anginal effect, the occurrence of pain and discomfort, and restriction of activity, even in the presence of continuing therapy and increased dosages. In such a case the subject should first be withdrawn from nitrate therapy for a period of time sufficient to restore efficacy of the medication and reversal of the tolerance. In this case the treating physician causes the subject to discontinue continuous nitrate therapy, but may still permit (at the discretion of the physician) sublingual nitroglycerin or nitroglycerin spray for the relief of acute angina attacks or as a prophylactic taken prior to exercise. Following a nitrate free period of between about 8 to about 24 hours, which can be achieved by a dose-free period of about 10 to about 12 hours in the case of immediate release nitroglycerin, about 14 hours for immediate release ISDN, and about 17 hours for immediate release ISMN, the nitrate therapy using the present delayed onset, extended release IS-5-MN oral dosage form is initiated. This is taken in the evening at bedtime. The delay in onset coupled with the tapering of release at the end of the dosing interval ensures that the subject obtains a therapeutic effect during the morning and throughout the day, but also has a sufficiently long drug free period at the end of the day to treat, prevent, reduce, reverse, and/or manage the nitrate tolerance. The drug free period coincides with the lowest risk period for angina attacks (nighttime and sleeping hours) for the safety and comfort of the subject. The treating physician will recognize the need to modify the dose according to the severity and frequency of symptoms. The recommended starting dose is 30 mg or 60 mg, once-daily. At the judgment of the treating physician the dose may be increased to 120 mg, once daily, after several days. Rarely, 240 mg daily may be required.

What is claimed is:

1. A method of treating at least one cardiovascular condition comprising administering, to a subject in need of such a treatment, a therapeutically effective amount of a delayed onset, extended release formulation of at least one isosorbide mononitrate (ISMN), or a pharmaceutically acceptable salt thereof, that exhibits the following characteristics upon administration to the subject:
i) a first phase, in which the plasma concentration of the at least one isosorbide mononitrate is maintained below a therapeutic level in the blood stream of the subject for at least 2 hours to about 12 hours following administration; followed by
ii) a second phase, in which the plasma concentration of the at least one isosorbide mononitrate in the blood stream of the subject is greater than or equal to the therapeutic level for about 6 to about 18 hours.

2. The method of claim 1, wherein the at least one cardiovascular condition comprises congestive heart failure and/or angina pectoris.

3. The method of claim 1, wherein the at least one cardiovascular condition comprises angina pectoris.

4. The method of claim 1, wherein the delayed onset, extended release formulation is administered orally.

5. The method of claim 1, wherein the delayed onset, extended release formulation is administered one time per day.

6. The method of claim 1, wherein the first phase lasts for at least about 2 to about 8 hours.

7. The method of claim 1, wherein the first phase lasts for at least about 2 to about 6 hours.

8. The method of claim 1, wherein the second phase lasts for at least about 6 to about 15 hours.

9. The method of claim 1, wherein the second phase lasts for at least about 6 to about 12 hours.

10. The method of claim 1, wherein the second phase lasts for at least about 8 to about 15 hours.

11. The method of claim 1, wherein the second phase lasts for at least about 8 to about 12 hours.

12. The method of claim 1, further comprising a third phase following the second phase, wherein the subject's plasma concentration of isosorbide mononitrate during the third phase is maintained below a therapeutically effective level.

13. The method of claim 12, wherein the third phase lasts for at least about 1 to about 8 hours.

14. The method of claim 12, wherein the third phase lasts for at least about 1 to about 6 hours.

15. The method of claim 12, wherein the third phase lasts for at least about 1 to about 4 hours.

16. The method of claim 1, wherein the delayed onset, extended release formulation comprises about 10 mg to about 150 mg of at least one isosorbide mononitrate.

17. The method of claim 16, wherein the maximal plasma concentration of isosorbide mononitrate in the blood of the subject occurs between about 3 hours and about 10 hours following administration.

18. The method of claim 16, wherein the delayed onset, extended release formulation comprises about 60 mg of IS-5-MN and the maximal plasma concentration of isosorbide mononitrate in the blood of the subject is between about 200 ng/ml and about 600 ng/ml.

19. The method of claim 16, wherein the delayed onset, extended release formulation comprises about 60 mg of IS-5-MN the bioavailability of the isosorbide mononitrate, as measured by AUCₐₗₗ is between about 4000 ng/ml/h and about 8000 ng/ml/h.

20. The method of claim 1, wherein the delayed onset, extended release formulation is administered to a subject at night.

21. The method of claim 20, wherein the second phase begins about 2 hours to about 8 hours following administration.

22. The method of claim 1, wherein the delayed onset, extended release formulation comprises an organic acid or base.

23. The method of claim 22, wherein the organic acid is chosen from among formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic, stearic, sulfanilic, alginic, and galacturonic acid, and mixtures thereof.

24. The method of claim 22, wherein the organic base is chosen from among sodium citrate, sodium succinate, sodium tartrate, potassium citrate, potassium tartrate, potassium succinate, and mixtures thereof.

25. The method of claim 1, wherein the at least one isosorbide mononitrate, or pharmaceutically acceptable salt thereof, is provided in the form of a powder.

26. The method of claim 1, wherein the delayed onset, extended release formulation is coated with at least one polymer.

27. The method of claim 1, wherein the delayed onset, extended release formulation is coated with at least one water-soluble polymer, water-insoluble polymer, or a combination thereof.

28. The method of claim 27, wherein the water soluble polymer is chosen from among polyvinyl alcohol, polyvinylpyrrolidone, methyl cellulose, hydroxypropyl cellulose, hydroxypropyl methyl cellulose, polyethylene glycol, and mixtures thereof.

29. The method of claim 27, wherein the water insoluble polymer is chosen from among ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene), poly(propylene), poly(ethylene oxide), poly(ethylene terephthalate), poly(vinyl isobutyl ether), poly(vinyl acetate), poly(vinyl chloride), polyurethane, and mixtures thereof.

30. The method of claim 1, wherein the delayed onset, extended release formulation is provided in an orally administrable dosage form.

31. The method of claim 1, wherein the delayed onset, extended release formulation is provided in a solid dosage form.

32. The method of claim 1, wherein the delayed onset, extended release formulation is provided as a tablet or capsule.

33. The method of claim 1, wherein the delayed onset, extended release formulation further comprises at least one pharmaceutically acceptable excipient.

34. The method of claim 33, wherein the excipient is a plasticizer, gelling agent, thickener, hardener, sealant, setting agent, suspending agent, surfactant, humectant, binder, carrier, diluent, or stabilizer.

35. The method of claim 34, wherein the stabilizer is fumaric acid.

36. The method of claim 34, wherein the binder is polyvinylpyrrolidone or ethyl cellulose.

37. The method of claim 1, wherein the delayed onset, extended release formulation further comprises one or more pharmaceutically active compounds.

38. The method of claim 1, wherein the delayed onset, extended release formulation is co-administered with one or more pharmaceutically active compounds.

39. The method of claim 1, wherein the at least one isosorbide mononitrate comprises IS-5-MN.

40. The method of claim 1, wherein the at least one isosorbide mononitrate comprises IS-2-MN.

41. The method of claim 39, wherein the delayed onset, extended release formulation comprises about 10, 20, 25, 30, 50, 60, 90, 100, or 120 mg of IS-5-MN.

42. The method of claim 39, wherein the delayed onset, extended release formulation comprises about 30, 60, 90, or 120 mg of IS-5-MN.

43. The method of claim 1, wherein a dose of the delayed onset, extended release formulation exhibits about 70% to about 130% of the bioavailability exhibited by the same dose of an immediate release formulation, when administered to a subject.

44. The method of claim 1, wherein a dose of the delayed onset, extended release formulation exhibits at least about 70% of the bioavailability exhibited by the same dose of an immediate release formulation, when administered to a subject.

45. The method of claim 1, wherein a dose of the delayed onset, extended release formulation exhibits at least about 80% of the bioavailability exhibited by the same dose of an immediate release formulation, when administered to a subject.

46. The method of claim 1, wherein a dose of the delayed onset, extended release formulation exhibits at least about 90% of the bioavailability exhibited by the same dose of an immediate release formulation, when administered to a subject.

47. A method of treating at least one cardiovascular condition comprising administering at nighttime, to a subject in need of such a treatment, a sustained release tablet formulation comprising at least one isosorbide mononitrate (ISMN), or a pharmaceutically acceptable salt thereof, wherein the formulation comprises an amount of ISMN that is ineffective to provide a therapeutic effect if administered in the morning.

48. A sustained release tablet formulation suitable for nighttime administration comprising at least one isosorbide mononitrate (ISMN), or a pharmaceutically acceptable salt thereof, wherein the formulation comprises an amount of ISMN that is less than the amount required to produce a therapeutic effect when administered in the morning.

49. A method of improving the bioavailability of ISMN in a subject comprising administering a sustained release tablet formulation comprising at least one ISMN at night.

50. A method of improving the bioavailability of ISMN in a subject comprising informing the subject to administer a sustained release tablet formulation comprising at least one ISMN at night, wherein the subject administers the formulation at night.
